(19) United States
(12) Patent Application Publication
(10) Pub. No.: US 2016/0149306 A1
(43) Pub. Date: May 26, 2016

(54) MICROSTRIP ANTENNA STRUCTURE AND MICROWAVE IMAGING SYSTEM USING THE SAME

(71) Applicant: METAL INDUSTRIES RESEARCH & DEVELOPMENT CENTRE, KAOHSIUNG (TW)

(72) Inventors: Cheng-Yuan HUNG, KAOHSIUNG CITY (TW); Min-Hang WENG, KAOHSIUNG CITY (TW); Siang-Wen LAN, KAOHSIUNG CITY (TW); Shoou-Jinn CHANG, TAINAN CITY (TW); Ru-Yuan YANG, KAOHSIUNG CITY (TW); Tsung-Chih YU, TAINAN CITY (TW)

(21) Appl. No.: 14/564,110

(22) Filed: Dec. 9, 2014

(30) Foreign Application Priority Data
Nov. 25, 2014 (TW) 103140845

(51) Int. Cl. H01Q 9/04; A61B 5/05

(52) U.S. Cl. CPC H01Q 9/0407; A61B 5/0507

(57) ABSTRACT

A microstrip antenna structure is provided, which includes a substrate, a ring microstrip line and a signal transmission port. The substrate has opposite first and second surfaces. The ring microstrip line is disposed on the first surface of the substrate. The ring microstrip line has a short coupling gap ranged between 0.004λ_{g} and 0.06λ_{g} for forming a radiation bandwidth with high selectivity, where λ_{g} represents a guided wavelength of an electromagnetic wave in the ring microstrip line corresponding to a center frequency of the radiation bandwidth. The signal transmission port is disposed on the second surface of the substrate. The signal transmission port penetrates through the substrate and is electrically connected to the ring microstrip line.

### RELATED APPLICATIONS

This application claims priority to Taiwan Application Serial Number 103140845, filed on Nov. 25, 2014, which is herein incorporated by reference.

### BACKGROUND

1. Field of Disclosure

The invention relates to a microstrip antenna structure and a microwave imaging system using the same, and more particularly, to microstrip antenna structure that can improve performance when operated in a high-frequency environment and a microwave imaging system using the same.

2. Description of Related Art

Microstrip antennas have the advantages such as light weight, small size and easy to manufacture and therefore, has been widely applied to radio communication devices such as mobile phones, navigators that demand of lightening and thinning. For the microstrip antennas, the generated radiation pattern, gain and polarity mainly depend on the structure and shape of the microstrip antenna. However, the known microstrip antenna is easily affected by conductor loss and dielectric loss of the substrate, such that excessive return losses may be encountered at a high-frequency operating environment, resulting in performance degradation of the radio communication device.

On the other hand, for microwave imaging systems of medical application, health conditions of inner organs of a human body can be detected through radio microwave signal transceived by a microwave coupling antenna. By the microwave image recovery technology applied to the microwave imaging system, non-invasive health diagnoses can be realized. Detection accuracy of diseased cells or tissues is in relation to the resolution and quality of scan images. However, because the microwave imaging system is usually operated in a high-frequency environment, if the known microstrip antenna is applied in the microwave imaging system, the resolution and quality of scan images may be degraded due to excessive return loss of the known microstrip antenna at high frequency, leading to lower the detection accuracy.

### SUMMARY

The objective of the invention is to provide a microstrip antenna structure. In this microstrip antenna structure, a short coupling gap structure is applied to the ring microstrip line thereof, coupling effect can be activated and the electrical length of the ring microstrip line can be improved in an operating environment with the frequency higher than 5 GHz, and the electromagnetic strength coupled by the ring microstrip line can be improved, thereby generating the characteristic of multiple resonances and forming lower return loss. The invention also provides a microwave imaging system with the abovementioned microstrip antenna structure, which can improve the image resolution and quality of an object.

One aspect of the invention is to provide a microstrip antenna structure. The microstrip antenna structure includes a substrate, a ring microstrip line and a signal transmission port. The substrate has opposite first and second surfaces. The ring microstrip line is disposed on the first surface of the substrate. The ring microstrip line has a short coupling gap ranged between 0.004λ_{g} and 0.06λ_{g} for forming a radiation bandwidth with high selectivity, where λ_{g} represents a guided wavelength of an electromagnetic wave in the ring microstrip line corresponding to a center frequency of the radiation bandwidth. The signal transmission port is disposed on the second surface of the substrate. The signal transmission port penetrates through the substrate and is electrically connected to the ring microstrip line.

In one or more embodiments, the ring microstrip line has a width ranged between 0.01λ_{g}, and 0.13λ_{g}.

In one or more embodiments, the ring microstrip line is a rectangular-shaped ring coaxial line, a rectangular-shaped ring coplanar waveguide line, a rectangular-shaped ring slotline or a rectangular-shaped ring stripline.

In one or more embodiments, the ring microstrip line includes titanium (Ti), cobaltum (Co), wolfram (W), hafnium (Hf), tantalum (Ta), molybdanium (Mo), chromium (Cr), agtentum (Ag), cuprum (Cu) or aluminium (Al).

In one or more embodiments, the substrate is a FR4 substrate, a RT/Duroid series substrate, an aluminum oxide substrate, a RO series substrate, a high temperature cofired ceramic (HTCC) substrate, a low temperature cofired ceramic (LTCC) substrate, a transparent conductive substrate or a semiconductor substrate. The RO series substrate includes magnesium oxide, calcium oxide, strontium oxide or barium oxide.

In one or more embodiments, the signal transmission port includes titanium, cobaltum, wolfram, hafnium, tantalum, molybdanium, chromium, agtentum, cuprum or aluminium.

In one or more embodiments, the microstrip antenna structure further includes a ground conductor. The ground conductor is disposed on the second surface of the substrate and is electrically insulated from the signal transmission port.

In one or more embodiments, the ground conductor defines an inner space, and the signal transmission port is located in the inner space.

In one or more embodiments, the ground conductor includes titanium, cobaltum, wolfram, hafnium, tantalum, molybdanium, chromium, agtentum, cuprum or aluminium.

Another aspect of the invention is to provide a microstrip antenna structure. The microstrip antenna structure includes a substrate, ring microstrip lines and signal transmission ports. The substrate has opposite first and second surfaces. The ring microstrip lines are disposed on the first surface of the substrate. Each of the ring microstrip lines has a short coupling gap ranged between 0.004λ_{g} and 0.06λ_{g} for forming a radiation bandwidth with high selectivity, where λ_{g} represents a guided wavelength of an electromagnetic wave in the ring microstrip line corresponding to a center frequency of the radiation bandwidth. The signal transmission ports are disposed on the second surface of the substrate. The signal transmission ports penetrate through the substrate and are respectively electrically connected to the ring microstrip lines.

In one or more embodiments, each two adjacent ones of the ring microstrip lines have a distance of 0.3λ_{g} and 0.5λ_{g} therebetween.

In one or more embodiments, each of the ring microstrip lines has a width ranged between 0.01λ_{g}, and 0.13λ_{g}.

In one or more embodiments, each of the ring microstrip lines is a rectangular-shaped ring coaxial line, a rectangular-shaped ring coplanar waveguide line, a rectangular-shaped ring slotline or a rectangular-shaped ring stripline.

In one or more embodiments, each of the ring microstrip lines includes titanium, cobaltum, wolfram, hafnium, tantalum, molybdanium, chromium, agtentum, cuprum or aluminium.

In one or more embodiments, the substrate is a FR4 substrate, a RT/Duroid series substrate, an aluminum oxide substrate, a RO series substrate, a HTCC substrate, a low temperature cofired ceramic LTCC substrate, a transparent conductive substrate or a semiconductor substrate. The RO series substrate includes magnesium oxide, calcium oxide, strontium oxide or barium oxide.

In one or more embodiments, each of the signal transmission ports includes titanium, cobaltum, wolfram, hafnium, tantalum, molybdanium, chromium, agtentum, cuprum or aluminium.

In one or more embodiments, the microstrip antenna structure further includes ground conductors. The ground conductors are disposed on the second surface of the substrate and are respectively electrically insulated from the signal transmission ports.

In one or more embodiments, each of the ground conductors defines an inner space, and the signal transmission ports are respectively located in the inner spaces.

In one or more embodiments, each of the ground conductors includes titanium, cobaltum, wolfram, hafnium, tantalum, molybdanium, chromium, agtentum, cuprum or aluminium.

Another aspect of the invention is to provide a microwave imaging system. The microwave imaging system includes a microwave scan unit, a microwave signal processing unit and a control and record unit. The microwave scan unit includes a transmitter and a receiver. The transmitter is used for generating an uniform electric field and radiating a microwave radio signal to an object, and the receiver is used for receiving the microwave radio signal penetrating through the object. The receiver includes a microstrip antenna structure. The microstrip antenna structure includes a substrate, at least one ring microstrip line and at least one signal transmission port. The at least one ring microstrip line is disposed on the first surface of the substrate. Each of the ring microstrip line has a short coupling gap ranged between 0.004λ_{g} and 0.06λ_{g} for forming a radiation bandwidth with high selectivity, where λ_{g} represents a guided wavelength of an electromagnetic wave in the ring microstrip line corresponding to a center frequency of the radiation bandwidth. The at least one signal transmission port is disposed on the second surface of the substrate. The at least one signal transmission port penetrates through the substrate and is respectively electrically connected to the at least one ring microstrip line. The microwave signal processing unit is electrically connected to the microwave scan unit. The microwave signal processing unit is used for inputting the microwave radio signal from the receiver and performing a dielectric parameter analysis and an image recovery analysis on the microwave radio signal. The control and record unit is electrically connected to the microwave scan unit and the microwave signal processing unit. The control and record unit is used for controlling the microwave scan unit, recording the microwave radio signal processed by the microwave signal processing unit and providing a data reading and writing function for the microwave signal processing unit.

It is to be understood that both the foregoing general description and the following detailed description are by examples, and are intended to provide further explanation of the disclosure as claimed.

### BRIEF DESCRIPTION OF THE DRAWINGS

The disclosure can be more fully understood by reading the following detailed description of the embodiment, with reference made to the accompanying drawings as follows:

FIG. 1 is a cross-sectional view of a microstrip antenna structure in accordance with some embodiments of the invention;

FIG. 2A is a top view of the microstrip antenna structure shown in FIG. 1;

FIG. 2B is a bottom view of the microstrip antenna structure shown in FIG. 1;

FIGS. 3A-3D illustrate electromagnetic strength distributions corresponding to various short coupling shown in FIG. 2A;

FIG. 4 illustrates the relationship between the electrical length and the short coupling gap of the ring microstrip line shown in FIG. 1;

FIG. 5 illustrates the relationship between the frequency and the retum loss of the microstrip antenna structure shown in FIG. 1;

FIG. 6A is a top view of a microstrip antenna structure in accordance with some embodiments of the invention;

FIG. 6B is a bottom view of a microstrip antenna structure in accordance with some embodiments of the invention; and

FIG. 7 is a schematic view of a microwave imaging system in accordance with some embodiments of the invention.

### DETAILED DESCRIPTION

In the following description, the disclosure will be explained with reference to embodiments thereof. However, these embodiments are not intended to limit the disclosure to any specific environment, applications or particular implementations described in these embodiments. Therefore, the description of these embodiments is only for the purpose of illustration rather than to limit the disclosure. In the following embodiments and attached drawings, elements not directly related to the disclosure are omitted from depiction; and the dimensional relationships among individual elements in the attached drawings are illustrated only for ease of understanding, but not to limit the actual scale.

It will be understood that, although the terms “first” and “second” may be used herein to describe various elements, components, regions, layers and/or sections, these elements, components, regions, layers and/or sections should not be limited by these terms. These terms are only used to distinguish one element, component, region, layer or section from another.

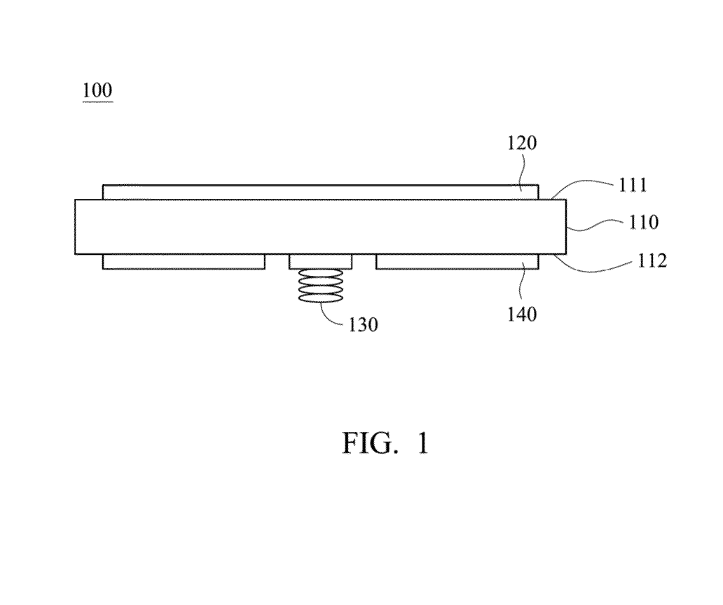

Referring to FIG. 1, FIG. 1 is a cross-sectional view of a microstrip antenna structure 100 in accordance with some embodiments of the invention. The microstrip antenna structure 100 is a single-feed antenna structure, which includes a substrate 110, a ring microstrip line 120, a signal transmission port 130 and a ground conductor 140. The substrate 110 may be a FR4 substrate, a RT/Duroid series substrate, an aluminum oxide substrate, a RO series substrate, a high temperature cofired ceramic (HTCC) substrate, a low temperature cofired ceramic (LTCC) substrate, a transparent conductive substrate or a semiconductor substrate or other similar substrates, in which the RO series substrate may include a material such as magnesium oxide, calcium oxide, strontium oxide, barium oxide or combinations thereof. The substrate 110 has opposite first and second surfaces 111, 112. The ring microstrip line 120 is disposed on the first surface 111, while the signal transmission port 130 is disposed on the second surface 112.

The ring microstrip line 120 forms a high-selective radiation bandwidth at the first surface 111. In this embodiment, the ring microstrip line 120 is rectangular-shaped and is a ring coaxial line, a ring coplanar waveguide line, a ring slotline or a ring stripline. In addition, the ring microstrip line 120 may include a metal such as titanium (Ti), cobaltum (Co), wolfram (W), hafnium (Hf), tantalum (Ta), molybdanium (Mo), chromium (Cr), agtentum (Ag), cuprum (Cu), aluminium (Al) or a metal alloy including the abovementioned metals, but is not limited thereto.

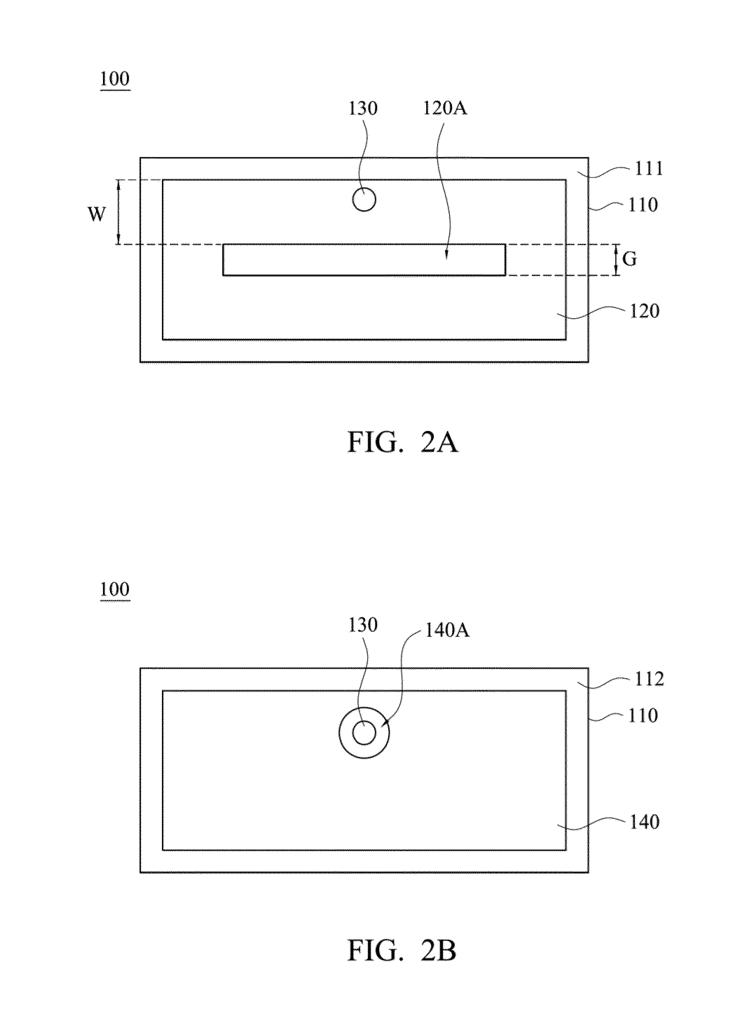

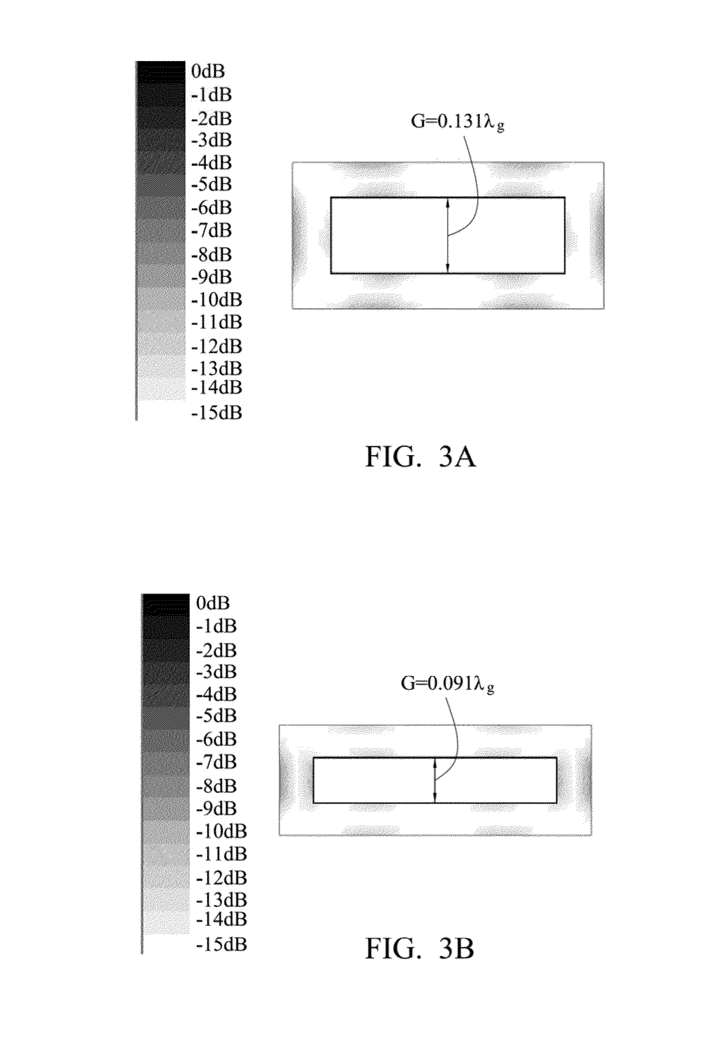

Referring to FIG. 2A, FIG. 2A is a top view of the microstrip antenna structure 100. In FIG. 2A, the ring microstrip line 120 defines a rectangular space 120A. The rectangular space 120A may be formed by performing lithography and etching processes. In some embodiments, the short coupling gap G of the rectangular space 120A is ranged between 0.004λ_{g} and 0.06λ_{g} (λ_{g} represents a guided wavelength of a center frequency of the radiation bandwidth generated by the ring microstrip line 120), and the short coupling gap G of the rectangular space 120A is used for activating coupling effect. Moreover, in some embodiments, the width W of the ring microstrip line 120 is ranged between 0.01λ_{g} and 0.13λ_{g}.

The signal transmission port 130 penetrates through the substrate 110 and is electrically connected to the ring microstrip line 120, which is used for conducting the signal received by the ring microstrip line 120. In some embodiments, the signal transmission port 130 may include a SMA plug for transmitting the signal from the ring microstrip line 120 to another place through an external cable. The signal transmission port 130 may include a metal such as titanium, cobaltum, wolfram, hafnium, tantalum, molybdanium, chromium, agtentum, cuprum, aluminium or a metal alloy including the abovementioned metals, but is not limited thereto. In some embodiments, the signal transmission port 130 includes the same material as the ring microstrip line 120.

The ground conductor 140 is disposed on the second surface 112 of the substrate 110. The ground conductor 140 may include a metal such as titanium, cobaltum, wolfram, hafnium, tantalum, molybdanium, chromium, agtentum, cuprum, aluminium or a metal alloy including the abovementioned metals, but is not limited thereto. In some embodiments, the ground conductor 140 includes the same material as the ring microstrip line 120 and/or the signal transmission port 130.

Referring to FIG. 2B, FIG. 2B is a bottom view of the microstrip antenna structure 100. In FIG. 2A, the ground conductor 140 defines a space 140A, and the signal transmission line 130 is located in the space 140A. In the planar direction of the microstrip antenna structure 100, a predetermined gap exists between the signal transmission port 130 and the ground conductor 140, such that the signal transmission port 130 and the ground conductor 140 are electrically insulated.

It should be noted that, the signal transmission port 130 may be disposed at any side of the ring microstrip line 120 based on various design demands, but is not limited at the place illustrated in FIG. 2.

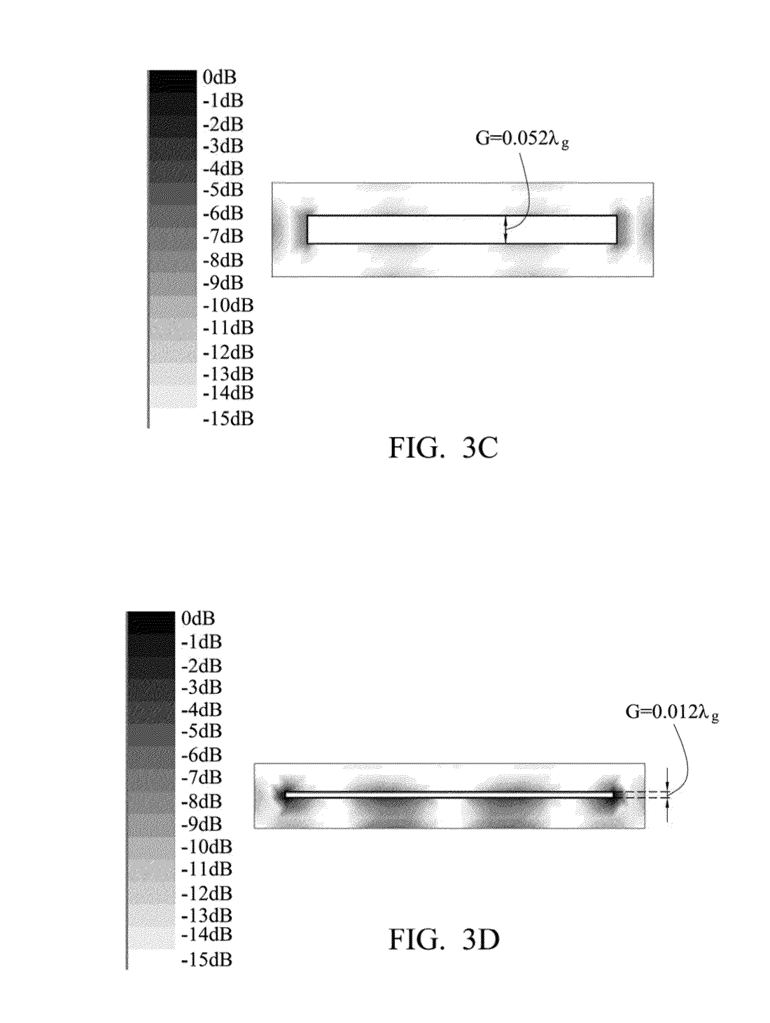

FIGS. 3A-3D illustrate electromagnetic strength distributions corresponding to the short coupling gaps of 0.131λ_{g}, 0.091λ_{g}, 0.052λ_{g} and 0.012λ_{g} respectively when the microstrip antenna structure is operated at the center frequency of 9.2 GHz. In FIGS. 3A-3D, the place with relatively dark color indicates that the electromagnetic strength is relatively strong and, oppositely, the place with relatively light color indicates that the electromagnetic strength is relatively weak, and the place with the darkest color represents that the electromagnetic strength is 120 A/m. As can be seen by comparing FIGS. 3A-3D, the guided wavelength λ_{g} of the ring microstrip line 120 with the short coupling gap G of 0.012λ_{g} is shorter, such that the electrical length of the ring microstrip line 120 increases correspondingly. In addition, the electromagnetic strength generated by the ring microstrip line 120 with the short coupling gap G of 0.012λ_{g} is the highest. As can be seen from the above, by reducing the short coupling gap G of the ring microstrip line 120, the electrical length of the ring microstrip line 120 can be enlarged, and the resonance energy of the ring microstrip line 120 can be improved, so as to generate the characteristic of multiple resonances and reduce the return loss of the microstrip antenna structure 100.

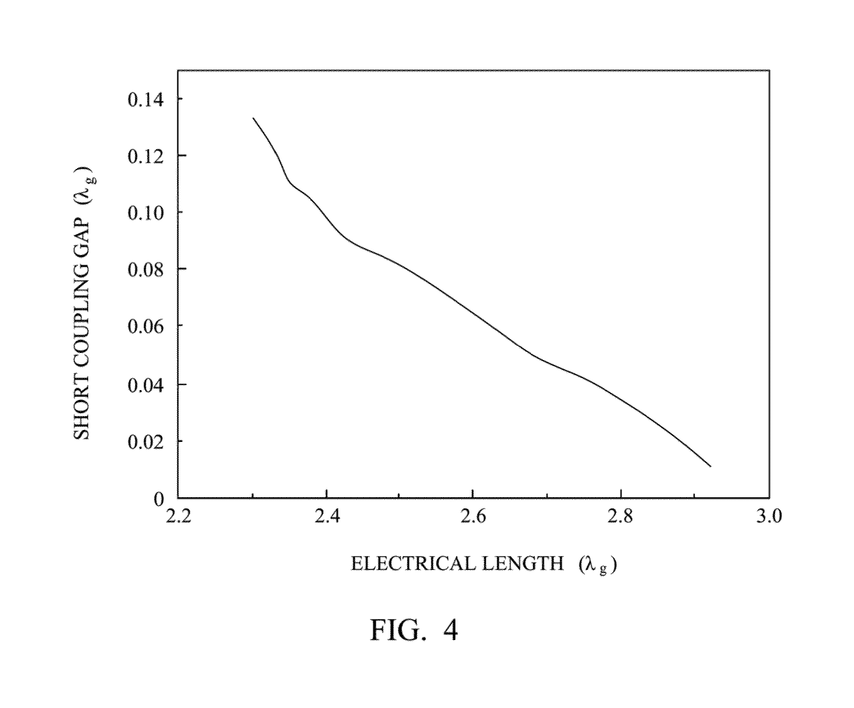

FIG. 4 illustrates the relationship between the electrical length and the short coupling gap G of the ring microstrip line 120. As can been seen from FIG. 4, when the shout coupling gap G is reduced, the electrical length of the ring microstrip line 120 increases correspondingly, which meets the simulation results as illustrated in FIGS. 3A-3D. Therefore, for design of the microstrip antenna structure 100, the short coupling gap G can be determined based on the desired electrical length.

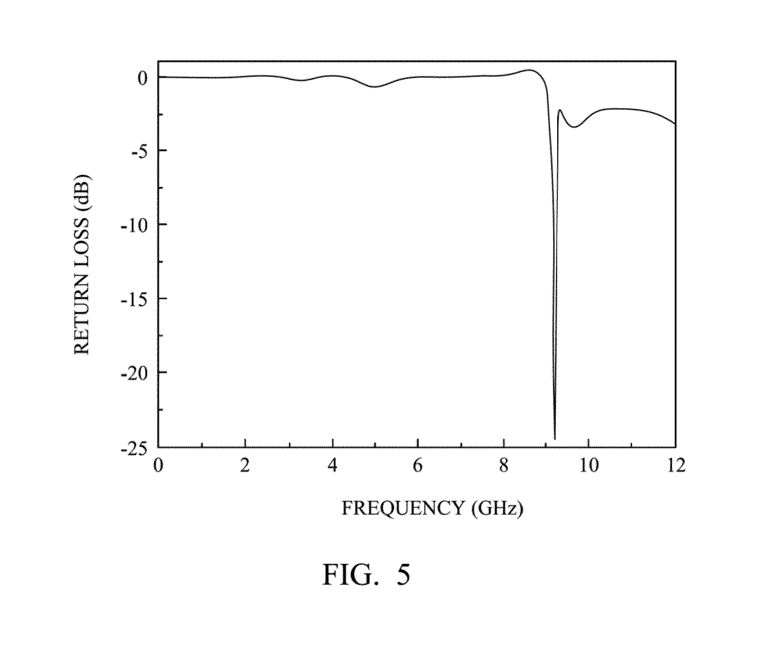

FIG. 5 illustrates the relationship between the frequency and the return loss of the microstrip antenna structure 100. The substrate 110 used for the microstrip antenna structure 100 is a FR4 substrate with the dielectric constant of 4.4 F/m, the thickness of 1.6 mm and the loss tangent of 0.025. The size of the ring microstrip line 120 is 0.16λ_{g}×1.51λ_{g}, and the center frequency of the radiate bandwidth generated by the ring microstrip line 120 is 9.2 GHz. As can be seen from FIG. 5, the return loss of the microstrip antenna structure 100 can be reduced to be near −25 dB at the frequency of 9.2 GHz.

As can be seen from the above, when the microstrip antenna structure 100 of the invention is operated in a high-frequency environment with the frequency higher than 5 GHz, relatively low return loss can be obtained, so as to improve the performance of signal-to-noise (SNR) ratio. Therefore, the microstrip antenna structure 100 of the invention is suitable for being applied in the radio communication devices that need to be operated in a high-frequency environment. On the other hand, the microstrip antenna structure 100 of the invention has the advantage of small size, and therefore, the manufacture cost can be reduced, the manufacture process can be simplified, and the difficulty of integrating the microstrip antenna structure 100 into a radio communication device can be reduced.

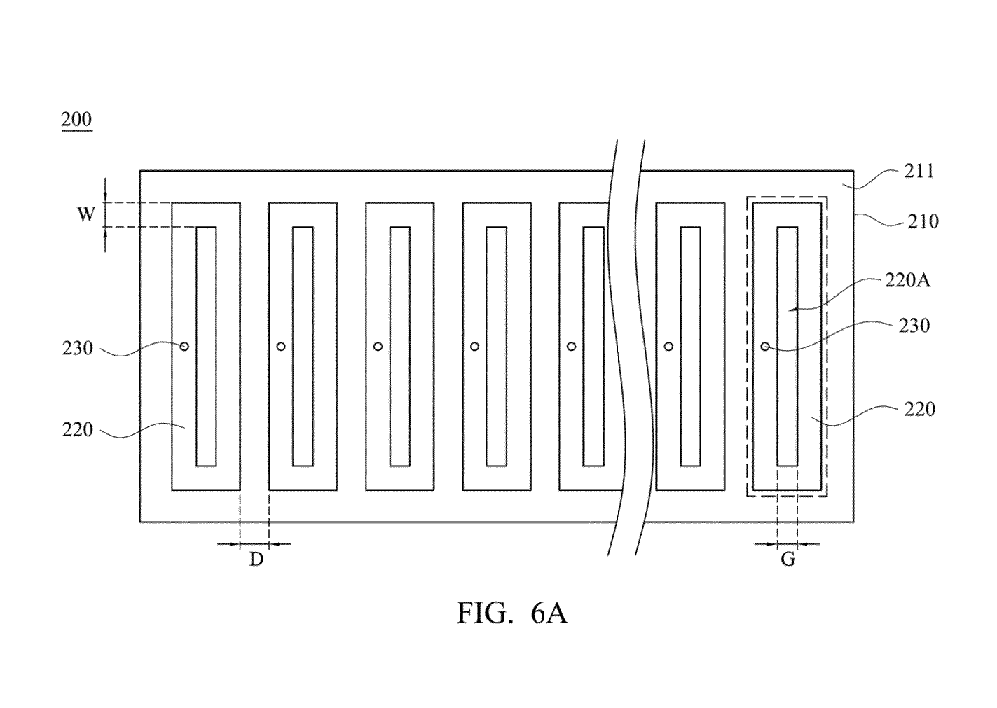

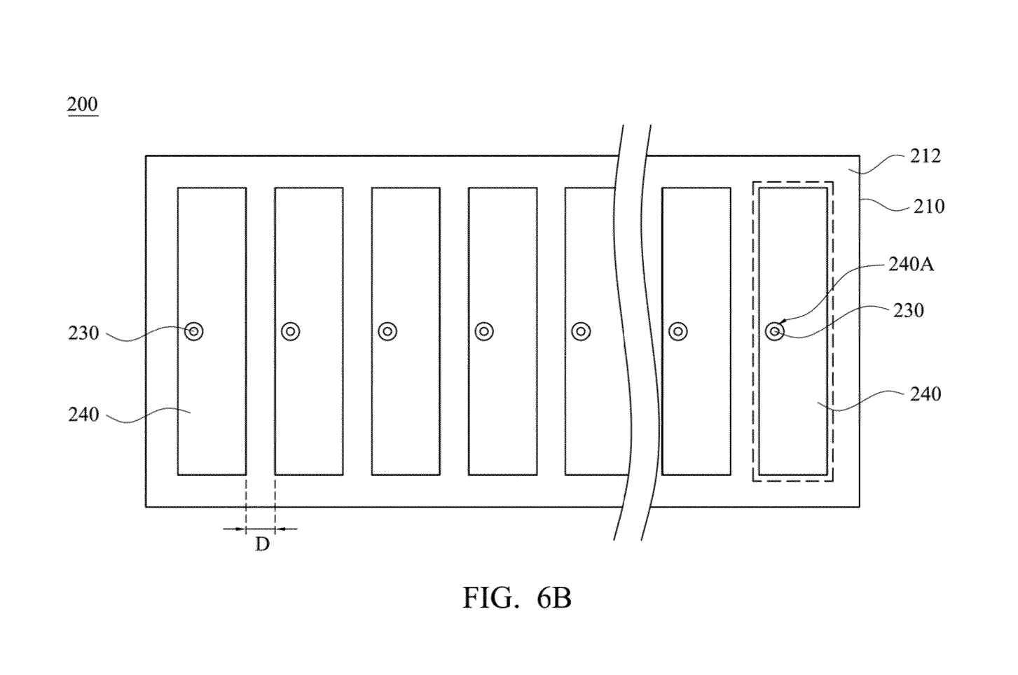

Referring to FIGS. 6A and 6B simultaneously, FIGS. 6A and 6B are top and bottom views of a microstrip antenna structure 200 respectively in accordance with some embodiments of the invention. The microstrip antenna structure 200 includes a substrate 210, ring microstrip lines 220, signal transmission ports 230 and ground conductors 240. The substrate 210 may be a FR4 substrate, a RT/Duroid series substrate, an aluminum oxide substrate, a RO series substrate, a HTCC substrate, a LTCC substrate, a transparent conductive substrate or a semiconductor substrate or other similar substrates, in which the RO series substrate may include a material such as magnesium oxide, calcium oxide, strontium oxide, barium oxide or combinations thereof. The substrate 210 has opposite first and second surfaces 211, 212. The ring microstrip lines 220 are disposed on the first surface 211, while the signal transmission ports 230 are disposed on the second surface 212. Each of the signal transmission ports 230 penetrates through the substrate 210 and is electrically connected to the corresponding ring microstrip line 220. The microstrip antenna structure 200 includes antenna units (as labeled by dashed lines in FIGS. 6A and 6B), and each of the antenna units includes one of the ring microstrip lines 220 and the corresponding signal transmission port 230 and ground conductor 240.

The ring microstrip lines 220 form a high-selective radiation bandwidth together at the first surface 211. In this embodiment, each of the ring microstrip lines 220 is rectangular-shaped, and the ring microstrip lines 220 may be one of a ring coaxial line, a ring coplanar waveguide line, a ring slotline a ring stripline respectively. Each of the ring microstrip lines 220 defines a rectangular space 220A. In some embodiments, the short coupling gap G of the ring microstrip line 220 is ranged between and 0.004λ_{g} and 0.06λ_{g} for activating coupling effect. In addition, each of the ring microstrip lines 220 may include a metal such as titanium, cobaltum, wolfram, hafnium, tantalum, molybdanium, chromium, agtentum, cuprum, aluminium or a metal alloy including the abovementioned metals, but is not limited thereto. In the microstrip antenna structure 200, the distance D between two adjacent ring microstrip lines 220 is ranged between 0.3λ_{g} and 0.5λ_{g}. Preferably, the distance D between two adjacent ring microstrip lines 220 is about 0.45λ_{g}. In some embodiments, the width W of each of the ring microstrip lines 220 is ranged between 0.01λ_{g} and 0.13λ_{g}.

Each of the signal transmission ports 230 penetrates through the substrate 210 and is electrically connected to the ring microstrip line 220 of the same antenna unit, which is used for conducting the signal received by the ring microstrip line 220. Each of the signal transmission ports 230 may include a metal such as titanium, cobaltum, wolfram, hafnium, tantalum, molybdanium, chromium, agtentum, cuprum, aluminium or a metal alloy including the abovementioned metals, but is not limited thereto. In some embodiments, the signal transmission ports 230 include the same material as the ring microstrip lines 220.

In each antenna unit, the ground conductor 240 defines a space 240A, and the signal transmission line 230 is located in the space 240A. In the planar direction of the microstrip antenna structure 200, a predetermined gap exists between the signal transmission port 230 and the ground conductor 240, such that the signal transmission port 230 and the ground conductor 240 are electrically insulated.

One feature of the microstrip antenna structure 200 is that, by the disposal of multiple antenna units and the design of the ring microstrip line 220 in each antenna unit, the receiving power of the electromagnetic signal can be enhanced, and the receiving range of the signal can be enlarged. When the microstrip antenna structure 200 is operated in a high-frequency environment with the frequency higher than 5 GHz, relatively low return loss can be obtained, so as to improve the performance of SNR ratio.

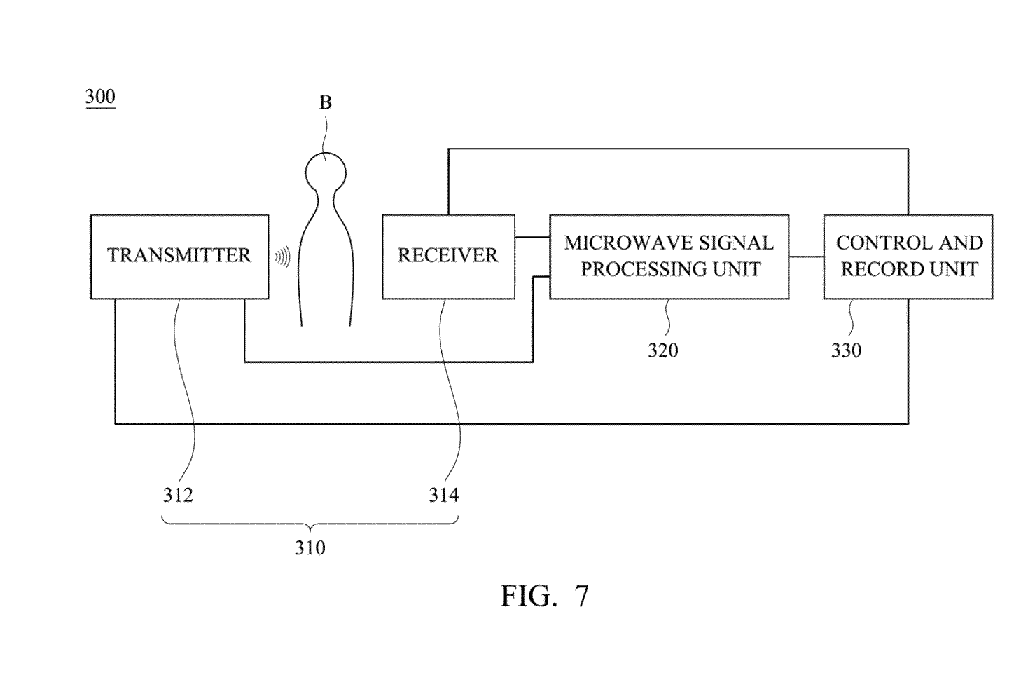

Referring to FIG. 7, FIG. 7 is a schematic view of a microwave imaging system 300 in accordance with some embodiments of the invention. The microwave imaging system 300 may be applied for microwave imaging application, such as microwave medical imaging application. For example, the microwave imaging system 300 can be applied for human brain detection or breast detection, but is not limited thereto.

In FIG. 7, the microwave imaging system 300 includes a microwave scan unit 310, a microwave signal processing unit 320 and a control and record unit 330. The microwave scan unit 310 includes a transmitter 312 and a receiver 314. The transmitter 312 is used for generating an uniform electric field and radiating a microwave radio signal to an object B, and the receiver 314 is used for receiving the microwave radio signal penetrating through the object B. In some embodiments, the planar size of the uniform electric filed generated by the transmitter 312 is larger than 900 cm². The receiver 314 may include the microstrip antenna structure 100 or 200 and, as such, when the microwave imaging system 300 is operated in a high-frequency environment (the operating frequency of the microwave imaging system is higher than 5 GHz), the performance of SNR ratio of the microwave imaging system 300 can be improved.

The microwave signal processing unit 320 is electrically connected to the microwave scan unit 310, which is used for inputting the microwave radio signal from the receiver 314 and performing a dielectric parameter analysis and an image recovery analysis on the microwave radio signal, so as to obtain a scan image of the object B.

The control and record unit 330 is electrically connected to the microwave scan unit 310 and the microwave signal processing unit 320, which is used for controlling the microwave scan unit 310, recording the microwave radio signal processed by the microwave signal processing unit 320 and providing a data reading and writing function for the microwave signal processing unit 320.

One feature of the microwave imaging system 300 is that, by applying the microstrip antenna structure 100 or 200, the return loss of the microwave imaging system 300 can be reduced when operated in an environment with the operating frequency higher than 5 GHz. Therefore, the microwave imaging system 300 of the invention can improve the image resolution and quality of an object, so as to improve the detection accuracy.

Although the disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the disclosure without departing from the scope or spirit of the disclosure. In view of the foregoing, it is intended that the disclosure cover modifications and variations of this disclosure provided they fall within the scope of the following claims.

What is claimed is:

1. A microstrip antenna structure, comprising:
a substrate having opposite first and second surfaces;
a ring microstrip line disposed on the first surface of the substrate, the ring microstrip line having a short coupling gap ranged between 0.004λ_{g} and 0.06λ_{g} for forming a radiation bandwidth with high selectivity, where λ_{g} represents a guided wavelength of an electromagnetic wave in the ring microstrip line corresponding to a center frequency of the radiation bandwidth; and
a signal transmission port disposed on the second surface of the substrate, the signal transmission port penetrating through the substrate and electrically connected to the ring microstrip line.

2. The microstrip antenna structure of claim 1, wherein the ring microstrip line has a width ranged between 0.01λ_{g} and 0.13λ_{g}.

3. The microstrip antenna structure of claim 1, wherein the ring microstrip line is a rectangular-shaped ring coaxial line, a rectangular-shaped ring coplanar waveguide line, a rectangular-shaped ring slotline or a rectangular-shaped ring stripline.

4. The microstrip antenna structure of claim 1, wherein the ring microstrip line comprises at least one material selected from the group consisting of titanium (Ti), cobaltum (Co), wolfram (W), hafnium (Hf), tantalum (Ta), molybdanium (Mo), chromium (Cr), agtentum (Ag), cuprum (Cu) and aluminium (Al).

5. The microstrip antenna structure of claim 1, wherein the substrate is a FR4 substrate, a RT/Duroid series substrate, an aluminum oxide substrate, a RO series substrate, a high temperature cofired ceramic (HTCC) substrate, a low temperature cofired ceramic (LTCC) substrate, a transparent conductive substrate or a semiconductor substrate;
wherein the RO series substrate comprises at least one material selected from the group consisting of magnesium oxide, calcium oxide, strontium oxide and barium oxide.

6. The microstrip antenna structure of claim 1, wherein the signal transmission port comprises at least one material selected from the group consisting of titanium, cobaltum, wolfram, hafnium, tantalum, molybdanium, chromium, agtentum, cuprum and aluminium.

7. The microstrip antenna structure of claim 1, further comprising:
a ground conductor disposed on the second surface of the substrate, the ground conductor electrically insulated from the signal transmission port.

8. The microstrip antenna structure of claim 7, wherein the ground conductor defines an inner space, and the signal transmission port is located in the inner space.

9. The microstrip antenna structure of claim 7, wherein the ground conductor comprises at least one material selected from the group consisting of titanium, cobaltum, wolfram, hafnium, tantalum, molybdanium, chromium, agtentum, cuprum and aluminium.

10. A microstrip antenna structure, comprising:
a substrate having opposite first and second surfaces;
a plurality of ring microstrip lines disposed on the first surface of the substrate, each of the ring microstrip lines having a short coupling gap ranged between 0.004λ_{g} and 0.06λ_{g} for forming a radiation bandwidth with high selectivity, where λ_{g} represents a guided wavelength of an electromagnetic wave in the ring microstrip line corresponding to a center frequency of the radiation bandwidth; and
a plurality of signal transmission ports disposed on the second surface of the substrate, the signal transmission ports penetrating through the substrate and respectively electrically connected to the ring microstrip lines.

11. The microstrip antenna structure of claim 10, wherein each two adjacent ones of the ring microstrip lines have a distance of 0.3λ_{g}, and 0.5λ_{g}, therebetween.

12. The microstrip antenna structure of claim 10, wherein each of the ring microstrip lines has a width ranged between 0.01λ_{g} and 0.13λ_{g}.

13. The microstrip antenna structure of claim 10, wherein each of the ring microstrip lines is a rectangular-shaped ring coaxial line, a rectangular-shaped ring coplanar waveguide line, a rectangular-shaped ring slotline or a rectangular-shaped ring stripline.

14. The microstrip antenna structure of claim 10, wherein each of the ring microstrip lines comprises at least one material selected from the group consisting of titanium, cobaltum, wolfram, hafnium, tantalum, molybdanium, chromium, agtentum, cuprum and aluminium.

15. The microstrip antenna structure of claim 10, wherein the substrate is a FR4 substrate, a RT/Duroid series substrate, an aluminum oxide substrate, a RO series substrate, a HTCC substrate, a low temperature cofired ceramic LTCC substrate, a transparent conductive substrate or a semiconductor substrate;
wherein the RO series substrate comprises at least one material selected from the group consisting of magnesium oxide, calcium oxide, strontium oxide and barium oxide.

16. The microstrip antenna structure of claim 10, wherein each of the signal transmission ports comprises at least one material selected from the group consisting of titanium, cobaltum, wolfram, hafnium, tantalum, molybdanium, chromium, agtentum, cuprum and aluminium.

17. The microstrip antenna structure of claim 10, further comprising:
a plurality of ground conductors disposed on the second surface of the substrate, the ground conductors electrically insulated from the signal transmission ports.

18. The microstrip antenna structure of claim 17, wherein each of the ground conductors defines an inner space, and the signal transmission ports are respectively located in the inner spaces.

19. The microstrip antenna structure of claim 17, wherein each of the ground conductors comprises at least one material selected from the group consisting of titanium, cobaltum, wolfram, hafnium, tantalum, molybdanium, chromium, agtentum, cuprum and aluminium.

20. A microwave imaging system, comprising:
a microwave scan unit having a transmitter and a receiver, the transmitter for generating an uniform electric field and radiating a microwave radio signal to an object, and the receiver for receiving the microwave radio signal penetrating through the object, wherein the receiver comprises a microstrip antenna structure, the microstrip antenna structure comprising:
a substrate having opposite first and second surfaces;
at least one ring microstrip line disposed on the first surface of the substrate, each of the at least one ring microstrip line having a short coupling gap ranged between 0.004λ_{g} and 0.06λ_{g} for forming a radiation bandwidth with high selectivity, where λ_{g} represents a guided wavelength of an electromagnetic wave in the ring microstrip line corresponding to a center frequency of the radiation bandwidth; and
at least one signal transmission port disposed on the second surface of the substrate, the at least one signal transmission port penetrating through the substrate and respectively electrically connected to the at least one ring microstrip line;
a microwave signal processing unit electrically connected to the microwave scan unit, the microwave signal processing unit for inputting the microwave radio signal from the receiver and performing a dielectric parameter analysis and an image recovery analysis on the microwave radio signal; and
a control and record unit electrically connected to the microwave scan unit and the microwave signal processing unit, the control and record unit for controlling the microwave scan unit, recording the microwave radio signal processed by the microwave signal processing unit and providing a data reading and writing function for the microwave signal processing unit.
